(19) United States
(12) Patent Application Publication
(10) Pub. No.: US 2025/0292887 A1
(43) Pub. Date: Sep. 18, 2025

(54) Systems, Method, And Computer Program Product For Associating Injector And Imager Protocols

(71) Applicant: Bayer Healthcare LLC, Whippany, NJ (US)

(72) Inventors: Barry SKIRBLE, Allison Park, PA (US); Sharon STANDISH, Pawleys Island, SC (US); Kai STRASDAS, Morris Plains, NJ (US); Sri SHRIRAM, Murrysville, PA (US); David GRIFFITHS, Pittsburgh, PA (US)

(21) Appl. No.: 18/859,536

(22) PCT Filed: Apr. 27, 2023
(86) PCT No.: PCT/US2023/020193

§ 371 Date: Oct. 23, 2024

Related U.S. Application Data
(60) Provisional application No. 63/336,492, filed on Apr. 29, 2022.
(60) Provisional application No. 63/358,783, filed on Jul. 6, 2022.

(51) Int. Cl. G16H 30/20; A61B 6/00; G16H 10/60; G16H 20/17; G16H 40/63

(52) U.S. Cl. CPC G16H 30/20; A61B 6/481; G16H 10/60; G16H 20/17; G16H 40/63

(57) ABSTRACT

Disclosed herein are system, method, and computer program product embodiments for associating injector and imager protocols. For example, the method may include: receiving, from an imaging system including an imager configured to image a patient, imager protocol data associated with an imager protocol according to which the imaging system is configured to control imaging of the patient with the imager, the imaging system being in communication with an injection system including an injector configured to deliver fluid and/or imaging agent to the patient; receiving, via at least one user interface, at least one of a selection, a programming, a modification, or any combination thereof of an injector protocol according to which the injection system is configured to control delivery of at least one fluid and/or imaging agent to the patient with the injector; and associating, in at least one database, the imager protocol with the injector protocol.

### CROSS-REFERENCE TO RELATED APPLICATIONS

This application claims the benefit of U.S. Provisional Application Nos. 63/336,492, filed Apr. 29, 2022, and 63/358,783, filed Jul. 6, 2022, the disclosures of which are hereby incorporated by reference in their entirety.

### BACKGROUND

### 1. Field

This disclosure relates generally to medical imaging, and in some non-limiting embodiments or aspects, to associating injector and imager protocols.

### 2. Technical Considerations

Operation and coordination of existing imaging devices and fluid delivery systems may occur in a sequential order and involve interdependencies and manual synchronization efforts including redundant, often manual entry of input values, such as patient selection, imager acquisition parameters, such as computed tomography (CT) tube voltage, patient weight, imager and injector protocol selection, and contrast media concentration. This manual selection and data entry may cause: errors in the input values, delays in the procedure or exam workflow, distractions from clinical tasks, exam quality issues, safety risks, and/or the like. Further, such manual selection and data entry may be regarded as less fulfilling administrative tasks by a radiographer/technologist. Moreover, complexity and time pressures may compromise the degree of exam personalization for a patient with potential implications for image quality and adherence to the “As Low As Reasonably Achievable” (ALARA) radiation safety principle.

### SUMMARY

Accordingly, provided are improved systems, devices, products, apparatus, and/or methods for associating injector and imager protocols.

According to some non-limiting embodiments or aspects, provided is a system, including: at least one processor coupled to a memory and configured to: receive, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient; receive, via at least one user interface, for the procedure for the patient, at least one of a selection, a programming, a modification, or any combination thereof of an injector protocol according to which the injection system is configured to control delivery of at least one fluid and/or imaging agent to the patient with the injector; and associate, in at least one database, the imager protocol with the injector protocol.

In some non-limiting embodiments or aspects, the at least one processor is further configured to: determine, from the at least one database, based on the imager protocol data associated with the imager protocol, at least one proposed injector protocol proposed to be used with the imager protocol in the procedure for the patient; and provide, via the at least one user interface, an option to select, program, or modify the at least one proposed injector protocol proposed for use with the imager protocol in the procedure for the patient.

In some non-limiting embodiments or aspects, the at least one proposed injector protocol is further determined, from the at least one database, based on a number of previous procedures for which the at least one proposed injector protocol was used with the imager protocol.

In some non-limiting embodiments or aspects, the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

In some non-limiting embodiments or aspects, the at least one processor is further configured to: automatically determine, based on at least one value of the at least one procedure specific imager parameter, at least one value of at least one procedure specific injector parameter associated with the at least one proposed injector protocol.

In some non-limiting embodiments or aspects, the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

In some non-limiting embodiments or aspects, automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

In some non-limiting embodiments or aspects, the at least one processor is further configured to: receive, via the at least one user interface, one or more values of one or more procedure specific injector parameters associated with the at least one proposed injector protocol; and automatically determine, based on the one or more values of the one or more procedure specific injector parameters, one or more values of one or more procedure specific imager parameters associated with the imager protocol.

In some non-limiting embodiments or aspects, automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

In some non-limiting embodiments or aspects, the at least one user interface provides, in a same display, a plurality of prompts for a plurality of procedure specific imager parameters associated with the imager protocol and a plurality of prompts for a plurality of procedure specific injector parameters associated with the at least one proposed injector protocol.

According to some non-limiting embodiments or aspects, provided is a computer-implemented method, including: receiving, with at least one processor, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient; receiving, with the at least one processor, via at least one user interface, for the procedure for the patient, at least one of a selection, a programming, a modification, or any combination thereof of an injector protocol according to which the injection system is configured to control delivery of at least one fluid and/or imaging agent to the patient with the injector; and associating, with the at least one processor, in at least one database, the imager protocol with the injector protocol.

In some non-limiting embodiments or aspects, the method further includes: determining, with the at least one processor, from the at least one database, based on the imager protocol data associated with the imager protocol, at least one proposed injector protocol proposed to be used with the imager protocol in the procedure for the patient; and providing, with the at least one processor, via the at least one user interface, an option to select, program, or modify the at least one proposed injector protocol proposed for use with the imager protocol in the procedure for the patient.

In some non-limiting embodiments or aspects, the at least one proposed injector protocol is further determined, from the at least one database, based on a number of previous procedures for which the at least one proposed injector protocol was used with the imager protocol.

In some non-limiting embodiments or aspects, the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

In some non-limiting embodiments or aspects, the method further includes: automatically determining, with the at least one processor, based on at least one value of the at least one procedure specific imager parameter, at least one value of at least one procedure specific injector parameter associated with the at least one proposed injector protocol.

In some non-limiting embodiments or aspects, the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

In some non-limiting embodiments or aspects, automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

In some non-limiting embodiments or aspects, the method further includes: receiving, with the at least one processor, via the at least one user interface, one or more values of one or more procedure specific injector parameters associated with the at least one proposed injector protocol; and automatically determining, with the at least one processor, based on the one or more values of the one or more procedure specific injector parameters, one or more values of one or more procedure specific imager parameters associated with the imager protocol.

In some non-limiting embodiments or aspects, wherein automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

In some non-limiting embodiments or aspects, the at least one user interface provides, in a same display, a plurality of prompts for a plurality of procedure specific imager parameters associated with the imager protocol and a plurality of prompts for a plurality of procedure specific injector parameters associated with the at least one proposed injector protocol.

According to non-limiting embodiments or aspects, provided is a computer program product comprising at least one non-transitory computer-readable medium including program instructions that, when executed by at least one processor, cause the at least one processor to: receive, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient; receive, via at least one user interface, for the procedure for the patient, at least one of a selection, a programming, a modification, or any combination thereof of an injector protocol according to which the injection system is configured to control delivery of at least one fluid and/or imaging agent to the patient with the injector; and associate, in at least one database, the imager protocol with the injector protocol.

In some non-limiting embodiments or aspects, the program instructions, when executed by the at least one processor, further cause the at least one processor to: determine, from the at least one database, based on the imager protocol data associated with the imager protocol, at least one proposed injector protocol proposed to be used with the imager protocol in the procedure for the patient; and provide, via the at least one user interface, an option to select, program, or modify the at least one proposed injector protocol proposed for use with the imager protocol in the procedure for the patient.

In some non-limiting embodiments or aspects, the at least one proposed injector protocol is further determined, from the at least one database, based on a number of previous procedures for which the at least one proposed injector protocol was used with the imager protocol.

In some non-limiting embodiments or aspects, the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

In some non-limiting embodiments or aspects, the program instructions, when executed by the at least one processor, further cause the at least one processor to: automatically determine, based on at least one value of the at least one procedure specific imager parameter, at least one value of at least one procedure specific injector parameter associated with the at least one proposed injector protocol.

In some non-limiting embodiments or aspects, the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

In some non-limiting embodiments or aspects, automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

In some non-limiting embodiments or aspects, the program instructions, when executed by the at least one processor, further cause the at least one processor to: receive, via the at least one user interface, one or more values of one or more procedure specific injector parameters associated with the at least one proposed injector protocol; and automatically determine, based on the one or more values of the one or more procedure specific injector parameters, one or more values of one or more procedure specific imager parameters associated with the imager protocol.

In some non-limiting embodiments or aspects, automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

In some non-limiting embodiments or aspects, the at least one user interface provides, in a same display, a plurality of prompts for a plurality of procedure specific imager parameters associated with the imager protocol and a plurality of prompts for a plurality of procedure specific injector parameters associated with the at least one proposed injector protocol.

Further non-limiting embodiments or aspects are set forth in the following numbered clauses:

Clause 1. A system, comprising: at least one processor coupled to a memory and configured to: receive, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient; receive, via at least one user interface, for the procedure for the patient, at least one of a selection, a programming, a modification, or any combination thereof of an injector protocol according to which the injection system is configured to control delivery of at least one fluid and/or imaging agent to the patient with the injector; and associate, in at least one database, the imager protocol with the injector protocol.

Clause 2. The system of clause 1, wherein the at least one processor is further configured to: determine, from the at least one database, based on the imager protocol data associated with the imager protocol, at least one proposed injector protocol proposed to be used with the imager protocol in the procedure for the patient; and provide, via the at least one user interface, an option to select, program, or modify the at least one proposed injector protocol proposed for use with the imager protocol in the procedure for the patient.

Clause 3. The system of any of clauses 1 or 2, wherein the at least one proposed injector protocol is further determined, from the at least one database, based on a number of previous procedures for which the at least one proposed injector protocol was used with the imager protocol.

Clause 4. The system of any of clauses 1-3, wherein the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

Clause 5. The system of any of clauses 1-4, wherein the at least one processor is further configured to: automatically determine, based on at least one value of the at least one procedure specific imager parameter, at least one value of at least one procedure specific injector parameter associated with the at least one proposed injector protocol.

Clause 6. The system of any of clauses 1-5, wherein the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

Clause 7. The system of any of clauses 1-6, wherein automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

Clause 8. The system of any of clauses 1-7, wherein the at least one processor is further configured to: receive, via the at least one user interface, one or more values of one or more procedure specific injector parameters associated with the at least one proposed injector protocol; and automatically determine, based on the one or more values of the one or more procedure specific injector parameters, one or more values of one or more procedure specific imager parameters associated with the imager protocol.

Clause 9. The system of any of clauses 1-8, wherein automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

Clause 10. The system of any of clauses 1-9, wherein the at least one user interface provides, in a same display, a plurality of prompts for a plurality of procedure specific imager parameters associated with the imager protocol and a plurality of prompts for a plurality of procedure specific injector parameters associated with the at least one proposed injector protocol.

Clause 11. A computer-implemented method, comprising: receiving, with at least one processor, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient; receiving, with the at least one processor, via at least one user interface, for the procedure for the patient, at least one of a selection, a programming, a modification, or any combination thereof of an injector protocol according to which the injection system is configured to control delivery of at least one fluid and/or imaging agent to the patient with the injector; and associating, with the at least one processor, in at least one database, the imager protocol with the injector protocol.

Clause 12. The computer-implemented method of clause 11, further comprising: determining, with the at least one processor, from the at least one database, based on the imager protocol data associated with the imager protocol, at least one proposed injector protocol proposed to be used with the imager protocol in the procedure for the patient; and providing, with the at least one processor, via the at least one user interface, an option to select, program, or modify the at least one proposed injector protocol proposed for use with the imager protocol in the procedure for the patient.

Clause 13. The computer-implemented method of any of clauses 11 or 12, wherein the at least one proposed injector protocol is further determined, from the at least one database, based on a number of previous procedures for which the at least one proposed injector protocol was used with the imager protocol.

Clause 14. The computer-implemented method of any of clauses 11-13, wherein the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

Clause 15. The computer-implemented method of any of clauses 11-14, further comprising: automatically determining, with the at least one processor, based on at least one value of the at least one procedure specific imager parameter, at least one value of at least one procedure specific injector parameter associated with the at least one proposed injector protocol.

Clause 16. The computer-implemented method of any of clauses 11-15, wherein the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

Clause 17. The computer-implemented method of any of clauses 11-16, wherein automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

Clause 18. The computer-implemented method of any of clauses 11-17, further comprising: receiving, with the at least one processor, via the at least one user interface, one or more values of one or more procedure specific injector parameters associated with the at least one proposed injector protocol; and automatically determining, with the at least one processor, based on the one or more values of the one or more procedure specific injector parameters, one or more values of one or more procedure specific imager parameters associated with the imager protocol.

Clause 19. The computer-implemented method of any of clauses 11-18, wherein automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

Clause 20. The computer-implemented method of any of clauses 11-19, wherein the at least one user interface provides, in a same display, a plurality of prompts for a plurality of procedure specific imager parameters associated with the imager protocol and a plurality of prompts for a plurality of procedure specific injector parameters associated with the at least one proposed injector protocol.

Clause 21. A computer program product comprising at least one non-transitory computer-readable medium including program instructions that, when executed by at least one processor, cause the at least one processor to: receive, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient; receive, via at least one user interface, for the procedure for the patient, at least one of a selection, a programming, a modification, or any combination thereof of an injector protocol according to which the injection system is configured to control delivery of at least one fluid and/or imaging agent to the patient with the injector; and associate, in at least one database, the imager protocol with the injector protocol.

Clause 22. The computer program product of clause 21, wherein the program instructions, when executed by the at least one processor, further cause the at least one processor to: determine, from the at least one database, based on the imager protocol data associated with the imager protocol, at least one proposed injector protocol proposed to be used with the imager protocol in the procedure for the patient; and provide, via the at least one user interface, an option to select, program, or modify the at least one proposed injector protocol proposed for use with the imager protocol in the procedure for the patient.

Clause 23. The computer program product of any of clauses 21 and 22, wherein the at least one proposed injector protocol is further determined, from the at least one database, based on a number of previous procedures for which the at least one proposed injector protocol was used with the imager protocol.

Clause 24. The computer program product of any of clauses 21-23, wherein the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

Clause 25. The computer program product of any of clauses 21-24, wherein the program instructions, when executed by the at least one processor, further cause the at least one processor to: automatically determine, based on at least one value of the at least one procedure specific imager parameter, at least one value of at least one procedure specific injector parameter associated with the at least one proposed injector protocol.

Clause 26. The computer program product of any of clauses 21-25, wherein the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

Clause 27. The computer program product of any of clauses 21-26, wherein automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

Clause 28. The computer program product of any of clauses 21-27, wherein the program instructions, when executed by the at least one processor, further cause the at least one processor to: receive, via the at least one user interface, one or more values of one or more procedure specific injector parameters associated with the at least one proposed injector protocol; and automatically determine, based on the one or more values of the one or more procedure specific injector parameters, one or more values of one or more procedure specific imager parameters associated with the imager protocol.

Clause 29. The computer program product of any of clauses 21-28, wherein automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

Clause 30. The computer program product of any of clauses 21-29, wherein the at least one user interface provides, in a same display, a plurality of prompts for a plurality of procedure specific imager parameters associated with the imager protocol and a plurality of prompts for a plurality of procedure specific injector parameters associated with the at least one proposed injector protocol.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of non-limiting embodiments or aspects of an environment in which systems, devices, products, apparatus, and/or methods, described herein, may be implemented;

FIG. 2 is a diagram of non-limiting embodiments or aspects of components of one or more devices and/or one or more systems of FIG. 1;

FIG. 3 is a flowchart of non-limiting embodiments or aspects of a process for associating injector and imager protocols; and

FIG. 4 illustrates non-limiting embodiments or aspects of a user interface for associating injector and imager protocols.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the term “communication” may refer to the reception, receipt, transmission, transfer, provision, and/or the like, of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a PDA, and/or other like devices. A computing device may also be a desktop computer or other form of non-mobile computer.

As used herein, the term “mobile device” may refer to one or more portable electronic devices configured to communicate with one or more networks. As an example, a mobile device may include a cellular phone (e.g., a smartphone or standard cellular phone), a portable computer (e.g., a tablet computer, a laptop computer, etc.), a wearable device (e.g., a watch, pair of glasses, lens, clothing, and/or the like), a personal digital assistant (PDA), and/or other like devices. The terms “client device” and “user device,” as used herein, refer to any electronic device that is configured to communicate with one or more servers or remote devices and/or systems. A client device or user device may include a mobile device, a network-enabled appliance (e.g., a network-enabled television, refrigerator, thermostat, and/or the like), a computer, an injection system, and/or any other device or system capable of communicating with a network.

As used herein, the term “server” and/or “processor” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, injectors, mobile devices, etc.) directly or indirectly communicating in the network environment may constitute a “system.” Reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

As used herein, the term “user interface” or “graphical user interface” refers to a generated display, such as one or more graphical user interfaces (GUIs) with which a user may interact, either directly or indirectly (e.g., through a keyboard, mouse, touchscreen, etc.).

Non-limiting embodiments or aspects of the present disclosure may receive, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient; receive, via at least one user interface, for the procedure for the patient, at least one of a selection, a programming, a modification, or any combination thereof of an injector protocol according to which the injection system is configured to control delivery of at least one fluid and/or imaging agent to the patient with the injector; and associate, in at least one database, the imager protocol with the injector protocol. In such an example, non-limiting embodiments or aspects of the present disclosure may determine, from the at least one database, based on the imager protocol data associated with the imager protocol, at least one proposed injector protocol proposed to be used with the imager protocol in the procedure for the patient; and provide, via the at least one user interface, an option to select, program, or modify the at least one proposed injector protocol proposed for use with the imager protocol in the procedure for the patient.

In this way, data (e.g., patient selection, patient weight, CT tube voltage, etc.) may be obtained automatically and/or entered only once by a user or operator. Accordingly, non-limiting embodiments or aspects of the present disclosure may provide for fewer data errors and better data consistency, less user fatigue due to fewer distractions from manual tasks allowing for more user focus for clinical tasks, more protocol personalization to the patient including automatic consideration and pre-calculation of protocol parameters, a faster workflow, and/or the like.

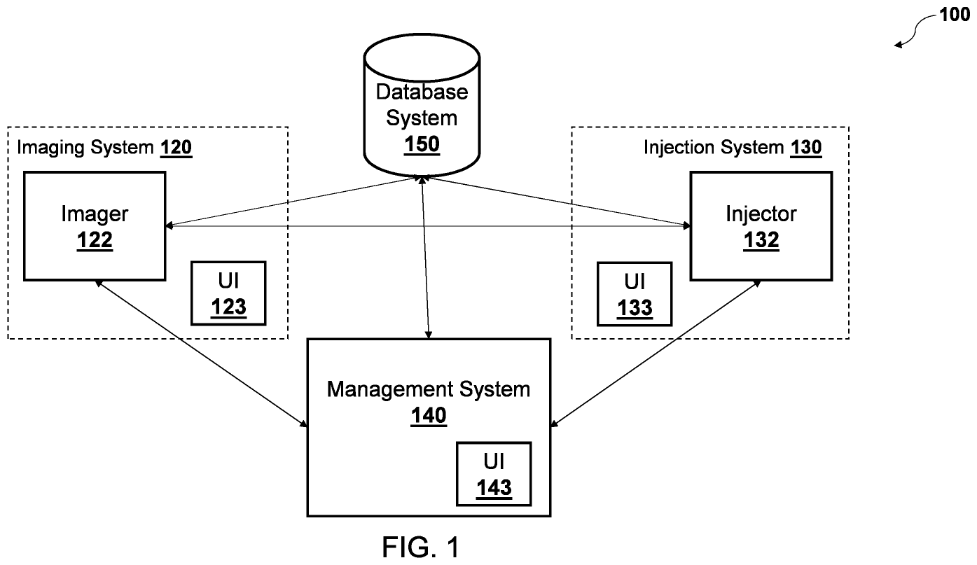

Referring now to FIG. 1, FIG. 1 is a diagram of an example environment 100 in which systems, devices, methods, and/or products described herein, may be implemented. As shown in FIG. 1, environment 100 may include imaging system 120 including imager or scanner 122, injection system 130 including injector 132, and/or management system 140, and/or database system 150. For example, environment 100 may include a hospital, an imaging center, a mobile imaging trailer, an emergency vehicle (e.g., an ambulance, etc.), a mobile imaging device (e.g., Hyperfine's Swoop® Portable MR Imaging System™, etc.), a mobile CT device, and/or the like.

Imaging system 120 including imager or scanner 122, injection system 130 including injector 132, management system 140, and/or database system 150 may interconnect (e.g., establish a connection to communicate, etc.) via wired connections, wireless connections, or a combination of wired and wireless connections (e.g., via communication network, etc.). For example, a communication network may include one or more wired and/or wireless networks. For example, a communication network may include a cellular network (e.g., a long-term evolution (LTE) network, a third generation (3G) network, a fourth generation (4G) network, a fifth generation (5G) network, a sixth generation (6G) network, a code division multiple access (CDMA) network, etc.), a public land mobile network (PLMN), a local area network (LAN), a wide area network (WAN), a metropolitan area network (MAN), a controller area network (CAN), a telephone network (e.g., the public switched telephone network (PSTN)), a private network, an ad hoc network, an intranet, the Internet, a fiber optic-based network, a cloud computing network, a short range wireless communication network (e.g., a Bluetooth network, a near field communication (NFC) network, etc.) and/or the like, and/or a combination of these or other types of networks.

Imaging system 120 may include one or more devices capable of receiving data and/or information from and/or communicating data and/or information to injection system 130, management system 140, and/or database system 150 (e.g., via a communication network, etc.). For example, imaging system 120 may include a computing device, such as a one or more computers, portable computers (e.g., tablet computers, etc.), mobile devices (e.g., cellular phones, smartphones, wearable devices, such as watches, glasses, lenses, and/or clothing, PDAs, and/or the like), a server, a group of servers, and/or other like devices. Imaging system 120 may include imager or scanner user interface 123 configured to receive data and/or information from and/or provide data and/or information to a user or operator.

Imaging system 120 may include one or more devices, software, and/or hardware configured to set up imager protocols (e.g., a scan time, a scan sequence, a scan energy level, etc.) and acquire non-contrast and/or contrast-enhanced scans of a patient. For example, imaging system 120 may include imager 122 configured to image a patient according to one or more imager protocols. As an example, imaging system 120 may include a magnetic resonance imaging (MRI) system; a computed tomography (CT) system; a positron emission tomography/computed tomography (PET/CT) system; a positron emission tomography/magnetic resonance (PET/MR) system; a single-photon emission computed tomography (SPECT) system; an ultrasound system; a radiopharmaceutical therapy (RPT) system, and/or the like. In such an example, imager 122 may include a magnetic resonance (MR) scanner; a computed tomography (CT) scanner; a positron emission tomography/computed tomography (PET/CT) scanner; a positron emission tomography/magnetic resonance (PET/MR) scanner; a single-photon emission computerized tomography (SPECT) scanner; an ultrasound scanner; a radiopharmaceutical therapy (RPT) scanner; and/or the like. As an example, imaging system 120 may include an imaging system as described in U.S. patent application Ser. No. 16/710,118, filed on Dec. 11, 2019, the disclosure of which is incorporated herein by reference in its entirety. In some non-limiting embodiments or aspects, imaging system 120 includes Siemens Healthineers' Somatom Go CT Systems, Siemens Healthineers' Magnetom Free. Star, Siemens Healthineers' syngo Virtual Cockpit, General Electric's Signa MR Systems, and/or the like. In some non-limiting embodiments or aspects, imaging system 120 includes a system for standardized MRI examinations with patient-centric scan workflow adaptations as described by U.S. patent application Ser. No. 17/458,753, filed on Aug. 27, 2021, the disclosure of which is incorporated herein by reference in its entirety.

Injection system 130 may include one or more devices capable of receiving data and/or information from and/or communicating data and/or information to imaging system 120, management system 140, and/or database system 150 (e.g., via a communication network, etc.). For example, injection system 130 may include a computing device, such as a one or more computers, portable computers (e.g., tablet computers, etc.), mobile devices (e.g., cellular phones, smartphones, wearable devices, such as watches, glasses, lenses, and/or clothing, PDAs, and/or the like), a server, a group of servers, and/or other like devices.

Injection system 130 may include one or more devices, software, and/or hardware configured to set up one or more injector protocols and deliver one or more fluids (e.g., saline, a contrast agent, etc.) and/or imaging agents (e.g., a non-fluid based imaging agent, such as a CO₂ imaging agent for CO₂ angiography using X-Ray imaging, a Xenon imaging agent for hyperpolarized Xenon lung imaging using MRI, and/or the like, etc.) to a patient according to one or more injector protocols. An injector protocol commonly includes one or more phases, with each phase specifying the fluid and/or imaging agent and optionally fluid concentration and/or imaging agent to be injected, and two of the flow rate (or dose rate), volume, and duration of that phase of the injection (e.g., because volume injected=flow rate×duration, or the integral of flow rate over injection duration for more complicated protocols, there may be only two independent variables out of those three parameters). Other injection parameters which may be different for different phases or constant for all phases may include at least one of a ratio of fluids injected (e.g., a percent of contrast in a total fluid flow, etc.), pressure limits, flow rate limits, occlusion indications, or any combination thereof. Some injectors may be configured to have a time varying value of one, some, or all of the injection parameters. For example, injection system 130 may include injector 132 (e.g., a contrast media injector, a medical fluid injector, etc.) configured to deliver one or more fluids (e.g., saline, a contrast agent, etc.) to a patient according to one or more injector protocols. As an example, injection system 130 may be configured to inject, deliver, or administer contrast fluid including a contrast agent to a patient, and in some non-limiting embodiments or aspects, to inject or administer saline or other fluid to a patient before, during, or after administration of contrast fluid. Injection system 130 may include injector user interface 133 configured to receive data and/or information from and/or provide data and/or information to a user or operator.

In some non-limiting embodiments or aspects, injection system 130 may include one or more exemplary injection systems or injectors as are disclosed in: U.S. patent application Ser. No. 09/715,330, filed on Nov. 17, 2000, issued as U.S. Pat. No. 6,643,537; U.S. patent application Ser. No. 09/982,518, filed on Oct. 18, 2001, issued as U.S. Pat. No. 7,094,216; U.S. patent application Ser. No. 10/825,866, filed on Apr. 16, 2004, issued as U.S. Pat. No. 7,556,619; U.S. patent application Ser. No. 12/437,011, filed on May 7, 2009, issued as U.S. Pat. No. 8,337,456; U.S. patent application Ser. No. 12/476,513, filed on Jun. 2, 2009, issued as U.S. Pat. No. 8,147,464; and U.S. patent application Ser. No. 11/004,670, filed on Dec. 3, 2004, issued as U.S. Pat. No. 8,540,698, the disclosures of each of which are incorporated herein by reference in their entireties. In some non-limiting embodiments or aspects, injection system 130 may include the MEDRAD® Centargo CT Injection System, the MEDRAD® Stellant CT Injection System With Certegra® Workstation, the MEDRAD® Stellant FLEX CT Injection System, the MEDRAD® MRXperion MR Injection System, the MEDRAD® Mark 7 Arterion Injection System, the MEDRAD® Intego PET Infusion System, the MEDRAD® Spectris Solaris EP MR Injection System, and/or the like.

Management system 140 may include one or more devices capable of receiving data and/or information from and/or communicating data and/or information to imaging system 120, injection system 130, and/or database system 150 (e.g., via a communication network, etc.). For example, management system 140 may include a computing device, such as a one or more computers, portable computers (e.g., tablet computers, etc.), mobile devices (e.g., cellular phones, smartphones, wearable devices, such as watches, glasses, lenses, and/or clothing, PDAs, and/or the like), a server, a group of servers, and/or other like devices. As an example, management system 140 may be implemented by and/or associated with a hospital, an imaging center, a mobile imaging trailer, an emergency vehicle (e.g., an ambulance, etc.), a mobile imaging device (e.g., Hyperfine's Swoop® Portable MR Imaging System™ etc.), a portable or mobile CT device (e.g. Siemens Healthineers SOMATOM® On.site), a radiology information system (RIS), and/or the like. Management system 140 may include management system user interface 143 configured to receive data and/or information from and/or provide data and/or information to a user or operator.

Database system 150 may include one or more devices capable of receiving information and/or data from imaging system 120, injection system 130, and/or management system 140 (e.g., via a communication network, etc.) and/or communicating information and/or data to imaging system 120, injection system 130, and/or management system 140 (e.g., via a communication network 110, etc.). For example, database system 150 may include one or more data storage devices and/or one or more computing systems including one or more processors (e.g., one or more computing devices, one or more server computers, one or more mobile computing devices, etc.). As an example, database system 150 may include an electronic medical record (EMR) system; a radiology information system (RIS); a picture archiving and communication system (PACS); a hospital information system (HIS); a hospital enterprise information system; an insurance or payment information system; a national health information system; a cross location network; an imager protocol database; a fixed injector protocol database, a smart injector protocol database; an imaging/injector protocol mapping database; any combination thereof; and/or the like.

Imaging system 120, injection system 130, and/or management system 140 may be in communication with the one or more data storage devices of database system 150, which may be local or remote to one or more of imaging system 120, injection system 130, and/or management system 140. Imaging system 120, injection system 130, and/or management system 140 may be capable of receiving data and/or information from, storing data and/or information in, communicating data and/or information to, or searching data and/or information stored in the one or more data storage devices.

An imager protocol database may store one or more imager protocols including imager protocol parameters according to which imaging system 120 is configured to control imaging or scanning of a patient with imager 122. A fixed injector protocol database may store one or more fixed injector protocols including fixed injector protocol parameters according to which injection system 130 is configured to control delivery of at least one fluid and/or imaging agent to a patient in an injection with injector 132. A smart injector protocol database may store one or more smart injector protocols including smart injector protocol parameters according to which injection system 130 is configured to control delivery of at least one fluid and/or imaging agent to a patient in an injection with injector 132. An imaging/injector protocol mapping database may include one or more imager protocols mapped to or associated with one or more injector protocols (e.g., one or more fixed injector protocols, one or more smart injector protocols, etc.).

In some non-limiting embodiments or aspects, an imager protocol includes one or more scan or imaging times, one or more scan or imaging sequences, one or more voltage levels (e.g., a tube current level, a tube voltage level, a kVp, etc.), any combination thereof, and/or the like.

In some non-limiting embodiments or aspects, a fixed injector protocol includes (and/or consists of) a flow rate (e.g., a fixed flow rate, etc.) of at least one fluid to be delivered to a patient in at least one phase of an injection and a volume (e.g., a fixed volume, etc.) of the at least one fluid to be delivered to the patient in the at least one phase of the injection. For example, a fixed injector protocol may include (and/or consist of) a first fixed flow rate and a first fixed volume of a first fluid to be delivered to a patient in a first phase of an injection and a second fixed flow rate and a second fixed volume of a second fluid to be delivered to the patient in a second phase of the injection.

In some non-limiting embodiments or aspects, for example, for CT based imaging, a smart injector protocol includes an iodine dose/load (IDL)-based protocol or algorithm or an iodine delivery rate (IDR)-based protocol or algorithm. A smart injector protocol may include a flow rate of at least one fluid to be delivered to a patient in at least one phase of an injection, a volume of the at least one fluid to be delivered to the patient in the at least one phase of the injection, a contrast or imaging agent dose amount (e.g., an iodine load in mg, a gadolinium dose in mmol, etc.), a contrast or imaging agent dose rate (e.g., a iodine delivery rate in mg/s, a gadolinium delivery rate in mmol/s, etc.), a weight-based parameter, a patient identifier, a patient body surface area, a patient height, a patient body mass index (BMI), a patient sex, a patient cardiac output, a patient anatomy to be imaged, or any combination thereof. For example, a smart injector protocol can include a protocol for the delivery of a fluid volume over time where the fluid volume includes at least a volumetric amount of a first fluid (e.g., Fluid A), such as contrast agent, delivered at a flow rate that may vary over time and may be zero at certain times, and a volumetric amount of a second fluid (e.g., Fluid B), such as saline, which also may be delivered at a flow rate that may vary over time and may be zero at certain times. In some non-limiting embodiments or aspects, a smart injector protocol includes a smart injector protocol including one or more smart injector protocol parameters as described in International Patent Application Publication No. WO2022164831A1, published Aug. 4, 2022, the disclosure of which is hereby incorporated by reference in its entirety.

The number and arrangement of devices and systems shown in FIG. 1 is provided as an example. There may be additional devices and/or systems, fewer devices and/or systems, different devices and/or systems, or differently arranged devices and/or systems than those shown in FIG. 1. Furthermore, two or more devices and/or systems shown in FIG. 1 may be implemented within a single device and/or system, or a single device and/or system shown in FIG. 1 may be implemented as multiple, distributed devices and/or systems. Additionally or alternatively, a set of devices and/or systems (e.g., one or more devices or systems) of environment 100 may perform one or more functions described as being performed by another set of devices and/or systems of environment 100.

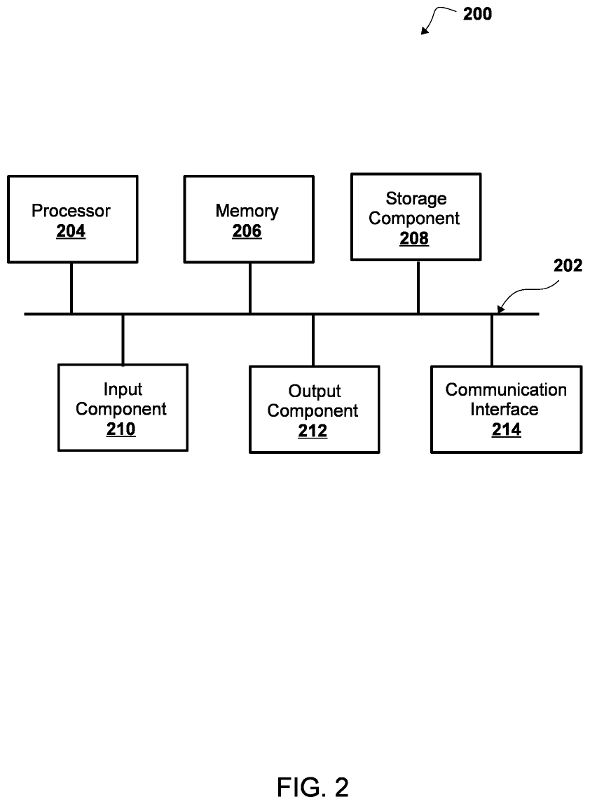

Referring now to FIG. 2, FIG. 2 is a diagram of example components of a device 200. Device 200 may correspond to one or more devices of imaging system 120, injection system 130, management system 140, and/or database system 150. In some non-limiting embodiments or aspects, one or more devices of imaging system 120, injection system 130, management system 140, and/or database system 150 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of non-transitory computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 (e.g., a central processing unit (CPU), a graphics processing unit (GPU), etc.) executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

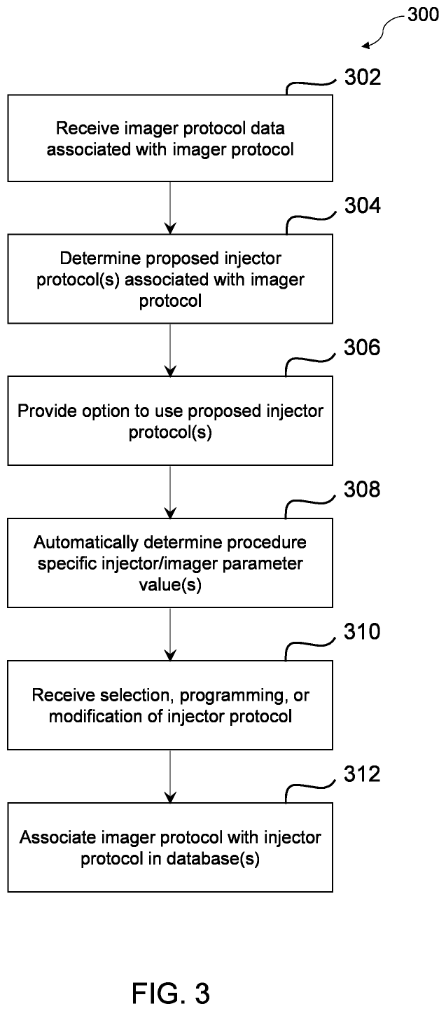

Referring now to FIG. 3, FIG. 3 is a flowchart of non-limiting embodiments or aspects of a process 300 for associating injector and imager protocols. In some non-limiting embodiments or aspects, one or more of the steps of process 300 may be performed (e.g., completely, partially, etc.) by injection system 130 (e.g., one or more devices of injection system 130, etc.). In some non-limiting embodiments or aspects, one or more of the steps of process 300 may be performed (e.g., completely, partially, etc.) by another device or a group of devices separate from or including injection system 130, such as imaging system 120 (e.g., one or more devices of imaging system 120, etc.), management system 140 (e.g., one or more devices of management system 140, etc.), and/or database system 150 (e.g., one or more devices of database system 150, etc.).

As shown in FIG. 3, at step 302, process 300 includes receiving imager protocol data associated with an imager protocol according to which an imaging system is configured to control imaging of a patient with an imager. For example, injection system 130 (and/or management system 140 and/or imaging system 120) may receive, from imaging system 120 including imager 122 configured to image a patient (and/or via at least one user interface, such as via imager or scanner user interface 123, via injector user interface 133, via management system user interface 143, and/or the like, etc.), for a procedure for a patient (e.g., a CT exam, etc.), imager protocol data associated with an imager protocol according to which imaging system 120 is configured to control imaging of the patient with imager 122. In such an example, imaging system 120 may be in communication with injection system 130 including injector 132 configured to deliver fluid and/or imaging agent to the patient (e.g., in direct communication with injection system 130, in indirect communication with injection system 130, such as via management system 140, and/or the like, etc.).

In some non-limiting embodiments or aspects, imager protocol data associated with an imager protocol includes at least one procedure specific imager parameter of the following procedure specific imager parameters: a patient name, a patient weight, a voltage level (e.g., a tube current level, a tube voltage level, a kilovoltage peak (kVp), etc.), an identifier of the imager protocol, a patient estimated glomerular filtration rate (eGFR), or any combination thereof.

As shown in FIG. 3, at step 304, process 300 includes determining, from at least one database, based on the imager protocol data associated with the imager protocol, at least one proposed injector protocol proposed to be used with the imager protocol. For example, injection system 130 (and/or management system 140 and/or imaging system 120) may determine, from at least one database (e.g., from database system 150, from an imaging/injector protocol mapping database, etc.), based on the imager protocol data associated with the imager protocol, at least one proposed injector protocol (e.g., a single proposed injector protocol, a plurality of proposed injector protocols, etc.) proposed to be used with the imager protocol in the procedure for the patient. As an example, database system 150 may include an imaging/injector protocol mapping database that includes one or more imager protocols mapped to or associated with one or more injector protocols (e.g., one or more fixed injector protocols, one or more smart injector protocols, etc.). In such an example, injection system 130 (and/or management system 140 and/or imaging system 120) may determine the at least one proposed injector protocol from one or more injector protocols that are mapped to or associated with the imager protocol associated with or identified by the imager protocol data.

In some non-limiting embodiments or aspects, the at least one proposed injector protocol may include each of the one or more injector protocols that are mapped to or associated with the imager protocol associated with or identified by the imager protocol data (e.g., a plurality of proposed injector protocols, etc.).

In some non-limiting embodiments or aspects, the at least one proposed injector protocol is further determined, from the at least one database, based on a number of previous procedures for which the at least one proposed injector protocol was used with the imager protocol. For example, injection system 130 (and/or management system 140 and/or imaging system 120) may rank a plurality of injector protocols that are mapped to or associated with the imager protocol in the imager/injector protocol mapping database according to number of previous procedures for which each injector protocol was used with the imager protocol. As an example, injection system 130 (and/or management system 140 and/or imaging system 120) may determine the at least one proposed injector protocol as the single highest or lowest ranked injector protocol or as a portion of the plurality of injector protocols that satisfy a threshold number of uses with the imager protocol.

In some non-limiting embodiments or aspects, injection system 130 (and/or management system 140 and/or imaging system 120) determines the at least one proposed injector protocol based on a machine learning technique (e.g., a pattern recognition technique, a data mining technique, a heuristic technique, a supervised learning technique, an unsupervised learning technique, etc.). For example, injection system 130 (and/or management system 140 and/or imaging system 120) may generate a model (e.g., an estimator, a classifier, a prediction model, etc.) based on a machine learning algorithm (e.g., a decision tree algorithm, a gradient boosted decision tree algorithm, a neural network algorithm, a convolutional neural network algorithm, etc.). In such an example, injection system 130 (and/or management system 140 and/or imaging system 120) may determine the at least one proposed injector protocol using the model.

Injection system 130 (and/or management system 140 and/or imaging system 120) may generate the model based on training data including imager protocol data associated with a plurality of imager protocols and/or injector protocol data associated with a plurality of injector protocols. For example, the model may be designed to receive, as an input, imager parameters associated with the imager protocols, procedure specific imager parameters associated with the imager protocols, injector parameters associated with the injector protocols, procedure specific injector parameters associated with the injector protocols, a number of previous procedures for which each injector protocol was used with each imager protocol, and/or the like, and provide, as an output, the at least one proposed injector protocol. As an example, injection system 130 (and/or management system 140 and/or imaging system 120) may analyze the training data using machine learning techniques to generate the model (e.g., a prediction model, a classification model, a recommendation model, etc.). The machine learning techniques may include, for example, supervised and/or unsupervised techniques, such as decision trees (e.g., gradient boosted decision trees), logistic regressions, artificial neural networks (e.g., convolutional neural networks), Bayesian statistics, learning automata, Hidden Markov Modeling, linear classifiers, quadratic classifiers, association rule learning, and/or the like. In some non-limiting embodiments or aspects, injection system 130 (and/or management system 140 and/or imaging system 120) generates or trains the model using the machine learning techniques to optimize an objective or loss function (e.g., an objective or loss function that depends on an output of the model and/or one or more labels for the input, etc.). In some non-limiting embodiments or aspects, injection system 130 (and/or management system 140 and/or imaging system 120) stores the trained model (e.g., stores the trained model for later use). In some non-limiting embodiments or aspects, injection system 130 (and/or management system 140 and/or imaging system 120) stores the trained model in a data structure (e.g., a database, a linked list, a tree, etc.). In some non-limiting embodiments or aspects, the data structure is located within injection system 130 (and/or management system 140 and/or imaging system 120) or external (e.g., remote from) injection system 130 (and/or management system 140 and/or imaging system 120).

As shown in FIG. 3, at step 306, process 300 includes providing, via at least one user interface, an option to select, program, and/or modify the at least one proposed injector protocol proposed for use with the imager protocol in the procedure for the patient. For example, injection system 130 (and/or management system 140 and/or imaging system 120) may provide, via at least one user interface (e.g., via imager or scanner user interface 123, via injector user interface 133, via a management system user interface 143, etc.), an option to select, program, and/or modify the at least one proposed injector protocol proposed for use with the imager protocol in the procedure for the patient.

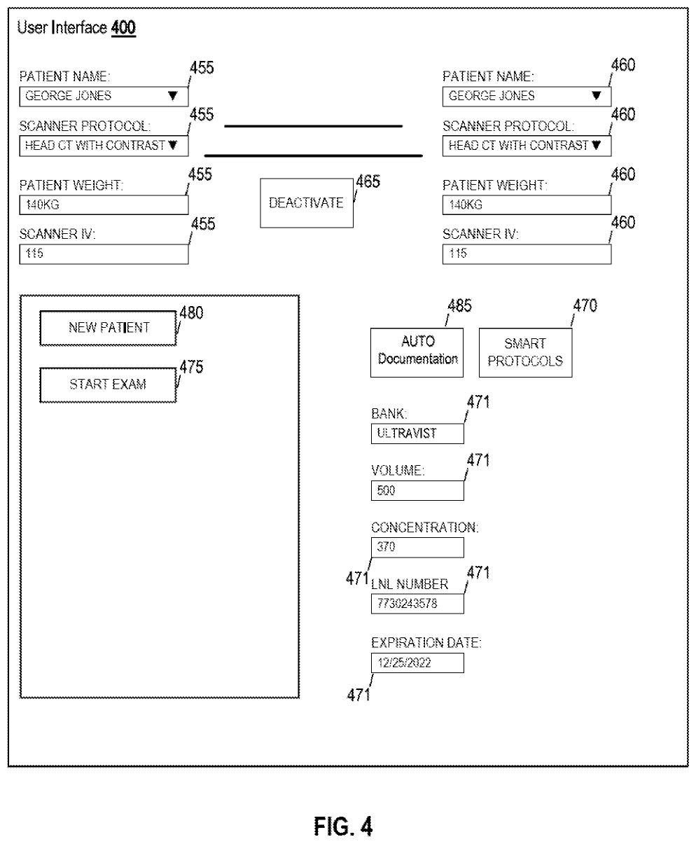

Referring also to FIG. 4, which illustrates non-limiting embodiments or aspects of a user interface 400 for associating injector and imager protocols, injection system 130 (and/or management system 140 and/or imaging system 120) may provide, in a same display (e.g., on imager or scanner user interface 123, on injector user interface 133, on management system user interface 143, etc.), a plurality of prompts or cells 455 for a plurality of procedure specific imager parameters associated with the imager protocol (e.g., a prompt or cell for a patient name, a prompt or cell for a scanner protocol name, a prompt or cell for a patient weight, a prompt or cell for a scanner kV level, etc.) and a plurality of prompts or cells 460 for a plurality of procedure specific injector parameters associated with the at least one proposed injector protocol (e.g., a prompt or cell for a patient name, a prompt or cell for a scanner protocol name, a prompt or cell for a patient weight, a prompt or cell for a scanner kV level, a prompt or cell for a contrast brand name, a prompt or cell for a fluid and/or imaging agent volume, a prompt or cell for a contrast concentration, a prompt or cell for a contrast lot number, a prompt or cell for a contrast expiration date, etc.).

As further shown in FIG. 4, user interface 400 for associating injector and imager protocols may include an activate/deactivate button 465 via which a user or operator may instruct injection system 130 (and/or management system 140 and/or imaging system 120) to automatically associate/disassociate the imager protocol with the at least one proposed injector protocol (or a user selected, programmed, and/or modified injector protocol, etc.) and/or to automatically populate/depopulate the corresponding prompts or cells as described in more detail herein. User interface 400 for associating injector and imager protocols may include a smart protocols button 470 via which a user or operator may instruct injection system 130 (and/or management system 140 and/or imaging system 120) to automatically display additional information and/or data associated with a smart protocol, such as a plurality of prompts or cells 471 for a plurality of smart protocol parameters that may be entered by the user or operator and/or automatically determined by the smart protocol. User interface 400 for associating injector and imager protocols may include a start procedure or exam button 475 via which a user or operator may instruct injection system 130 (and/or management system 140 and/or imaging system 120) to control imager 122 and/or injector 132 to initiate the procedure or exam for the patient (e.g., to initiate imaging of the patient, to initiate fluid and/or imaging agent delivery to the patient, etc.). User interface 400 for associating injector and imager protocols may include a new patient button 480 via which a user or operator may instruct injection system 130 (and/or management system 140 and/or imaging system 120) to automatically clear or reinitialize (e.g., to null or zero, etc.) all parameters and/or values associated with the imager protocol and/or the injector protocol. User interface 400 for associating injector and imager protocols may include an automated documentation button 485 via which a user or operator may instruct injection system 130 (and/or management system 140 and/or imaging system 120) to automatically store and/or document all parameters and/or values associated with the imager protocol and/or the injector protocol in association with the patient, the procedure.

As shown in FIG. 3, at step 308, process 300 includes automatically determining, based on at least one value of at least one procedure specific imager parameter, at least one value of at least one procedure specific injector parameter. For example, injection system 130 (and/or management system 140 and/or imaging system 120) may automatically determine, based on at least one value of the at least one procedure specific imager parameter, at least one value of at least one procedure specific injector parameter associated with the at least one proposed injector protocol.

Referring also to FIG. 4, injection system 130 (and/or management system 140 and/or imaging system 120) may automatically populate the prompts or cells 455 for the plurality of procedure specific imager parameters associated with the imager protocol with the imager data (e.g., with the at least one value of the at least one procedure specific imager parameter, etc.) and/or with information or data retrieved from database system 150 based on the imager data (e.g., a patient weight determined from a patient database in database system 150 based on a patient name known from the imager data, etc.).

Injection system 130 (and/or management system 140 and/or imaging system 120) may automatically determine the at least one value of the at least one procedure specific injector parameter based on the imager data, information or data associated with the at least one proposed injector protocol (or a user selected, programmed, and/or modified injector protocol, etc.), information or data retrieved from database system 150 based on the imager data and/or the at least one proposed injector protocol (or the user selected, programmed, and/or modified injector protocol, etc.), and/or information or data calculated using one or more algorithms (e.g., one or more smart protocol algorithms, etc.) based on the imager data, the information or data associated with the at least one proposed injector protocol (or the user selected, programmed, and/or modified injector protocol, etc.), and/or the information or data retrieved from database system 150. Injection system 130 (and/or management system 140 and/or imaging system 120) may automatically populate the prompts or cells 460 for the at least one procedure specific injector parameter with the determined at least one value of the at least one procedure specific injector parameter.

For example, injection system 130 (and/or management system 140 and/or imaging system 120) may automatically assign a patient name, a patient weight, an imager protocol name, and/or a voltage level associated with the imager protocol to corresponding procedure specific injector parameters associated with the at least one proposed injector protocol (or a user selected, programmed, and/or modified injector protocol, etc.). As an example, and as shown in FIG. 4, injection system 130 (and/or management system 140 and/or imaging system 120) may automatically populate, in the user interface, the prompts or cells 460 for the corresponding procedure specific injector parameter associated with the at least one proposed injector protocol (or a user selected, programmed, and/or modified injector protocol, etc.) with the patient name, the patient weight, the imager protocol name, and the voltage level associated with the imager protocol.

In some non-limiting embodiments or aspects, the at least one proposed injector protocol (or a user selected, programmed, and/or modified injector protocol, etc.) includes a smart injector protocol. For example, injection system 130 (and/or management system 140 and/or imaging system 120) may automatically determine the at least one value of the at least one procedure specific injector parameter associated with the at least one proposed injector protocol by calculating, based on the at least one value of the at least one procedure specific imager parameter (and/or based on the imager data, the information or data associated with the at least one proposed injector protocol (or the user selected, programmed, and/or modified injector protocol, etc.), and/or the information or data retrieved from database system 150), at least one smart injector protocol parameter of the following smart injector protocol parameters: a flow rate (or dose rate) of at least one fluid and/or imaging agent to be delivered to a patient in at least one phase of an injection, a volume of the at least one fluid and/or imaging agent to be delivered to the patient in the at least one phase of the injection, a contrast or imaging agent dose amount (e.g., an iodine load in mg, a gadolinium dose in mmol, etc.), a contrast or imaging agent dose rate (e.g., an iodine delivery rate in mg/s, a gadolinium delivery rate in mmol/s, etc.), a weight-based parameter, a patient identifier, a patient body surface area, a patient height, a patient body mass index (BMI), a patient sex, a patient cardiac output, a patient anatomy to be imaged (e.g., a liver, a heart, a brain, etc.), or any combination thereof. In some non-limiting embodiments or aspects, injection system 130 (and/or management system 140 and/or imaging system 120) may calculate, based on the at least one value of the at least one procedure specific imager parameter (and/or based on the imager data, the information or data associated with the at least one proposed injector protocol (or the user selected, programmed, and/or modified injector protocol, etc.), and/or the information or data retrieved from database system 150), at least one smart injector protocol parameter according to one or more of the equations or algorithms described in International Patent Application Publication No. WO2022164831A1, published Aug. 4, 2022, the disclosure of which is hereby incorporated by reference in its entirety.

In some non-limiting embodiments or aspects, injection system 130 (and/or management system 140 and/or imaging system 120) receives, via the at least one user interface, one or more values of one or more procedure specific injector parameters associated with the at least one proposed injector protocol; and automatically determines, based on the one or more values of the one or more procedure specific injector parameters, one or more values of one or more procedure specific imager parameters associated with the imager protocol. For example, injection system 130 (and/or management system 140 and/or imaging system 120) may automatically populate a prompt or cell for the one or more procedure specific imager parameters in the at least one user interface with the determined one or more values of the one or more procedure specific imager parameters. As an example, a user or operator may select, program, and/or modify one or more values of one or more procedure specific injector parameters associated with the at least one proposed injector protocol (e.g., after receiving the imager data and/or populating the user interface based thereon, etc.), and injection system 130 (and/or management system 140 and/or imaging system 120) may automatically update values of any procedure specific imager parameters affected by the selection, programming, and/or modification of the one or more procedure specific injector parameters. For example, if the user or operator changes the patient name associated with the at least one proposed injector protocol, injection system 130 (and/or management system 140 and/or imaging system 120) may automatically update the patient name associated with the imager protocol to the changed name.

As shown in FIG. 3, at step 310, process 300 includes receiving, via the at least one user interface, for the procedure for the patient, at least one of a selection, a programming, a modification, or any combination thereof of an injector protocol. For example, injection system 130 (and/or management system 140 and/or imaging system 120) may receive, via the at least one user interface (e.g., via imager or scanner user interface 123, via injector user interface 133, via management system user interface 143, etc.), for the procedure for the patient, at least one of a selection, a programming, a modification, or any combination thereof of an injector protocol (e.g., of the at least one proposed injector protocol, of an injector protocol different than the at least one proposed injector protocol, of an non-proposed injector protocol, etc.) according to which injection system 130 is configured to control delivery of at least one fluid and/or imaging agent to the patient with injector 132. As an example, a user or operator may select, program, and/or modify any injector protocol available at injection system 130 and/or database system 150 to be used with the imager protocol for the procedure for the patient.

As shown in FIG. 3, at step 312, process 300 includes associating, in the at least one database, the imager protocol with the injector protocol. For example, injection system 130 (and/or management system 140 and/or imaging system 120) may associate, in the at least one database (e.g., in database system 150, in an imaging/injector protocol mapping database, etc.), the imager protocol with the injector protocol. As an example, injection system 130 (and/or management system 140 and/or imaging system 120) may store, in the at least one database, the imager protocol and/or an identifier thereof in association with injector protocol and/or an identifier thereof.

In some non-limiting embodiments or aspects, and referring also to FIG. 4, injection system 130 (and/or management system 140 and/or imaging system 120) may associate (or disassociate), in the at least one database, the imager protocol with (or from) the injector protocol in response to an actuation of activate/deactivate button 465 in the at least one user interface. In some non-limiting embodiments or aspects, injection system 130 (and/or management system 140 and/or imaging system 120) may associate, in the at least one database, the imager protocol with the injector protocol in response initiating, conducting, and/or completing the procedure for the patient with the imager protocol and the injector protocol. For example, imaging system 120 may control imaging of the patient with imager 122 according to the imager protocol and injection system 130 may control delivery of the at least one fluid and/or imaging agent to the patient with injector 132 according to the injector protocol.

In this way, a library of associated protocols may be built over time through normal use of imaging system 120 and injection system 130 for patient procedures. The associated protocols may be maintained for a particular scanner—injector pair and/or shared across a plurality of scanner—injector pairs. Accordingly, non-limiting embodiments or aspects of the present disclosure may continuously build and refine a library or database of mappings or associations between imager protocols and injector protocols by tracking statistics associated with injector protocols used for a given scanner protocol, which may increase a likelihood of correct pre-selection or proposal of injector protocols for the user or operator. In such an example, an administrator may review the library or database of mappings or associations to manually generate or modify the mappings or associations.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A system, comprising:
at least one processor coupled to a memory and configured to:
receive, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient;
receive, via at least one user interface, for the procedure for the patient, at least one of a selection, a programming, a modification, or any combination thereof of an injector protocol according to which the injection system is configured to control delivery of at least one fluid and/or imaging agent to the patient with the injector; and
associate, in at least one database, the imager protocol with the injector protocol.

2. The system of claim 1, wherein the at least one processor is further configured to:
determine, from the at least one database, based on the imager protocol data associated with the imager protocol, at least one proposed injector protocol proposed to be used with the imager protocol in the procedure for the patient; and
provide, via the at least one user interface, an option to select, program, or modify the at least one proposed injector protocol proposed for use with the imager protocol in the procedure for the patient.

3. The system of claim 2, wherein the at least one proposed injector protocol is further determined, from the at least one database, based on a number of previous procedures for which the at least one proposed injector protocol was used with the imager protocol.

4. The system of claim 2, wherein the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

5. The system of claim 4, wherein the at least one processor is further configured to:
automatically determine, based on at least one value of the at least one procedure specific imager parameter, at least one value of at least one procedure specific injector parameter associated with the at least one proposed injector protocol.

6. The system of claim 5, wherein the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

7. The system of claim 5, wherein automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

8. The system of claim 5, wherein the at least one processor is further configured to:
receive, via the at least one user interface, one or more values of one or more procedure specific injector parameters associated with the at least one proposed injector protocol; and
automatically determine, based on the one or more values of the one or more procedure specific injector parameters, one or more values of one or more procedure specific imager parameters associated with the imager protocol.

9. The system of claim 8, wherein automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

10. The system of claim 5, wherein the at least one user interface provides, in a same display, a plurality of prompts for a plurality of procedure specific imager parameters associated with the imager protocol and a plurality of prompts for a plurality of procedure specific injector parameters associated with the at least one proposed injector protocol.

11. A computer-implemented method, comprising:
receiving, with at least one processor, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient;
receiving, with at least one processor, via at least one user interface, for the procedure for the patient, at least one of a selection, a programming, a modification, or any combination thereof of an injector protocol according to which the injection system is configured to control delivery of at least one fluid and/or imaging agent to the patient with the injector; and
associating, with the at least one processor, in at least one database, the imager protocol with the injector protocol.

12-20. (canceled)

21. A computer program product comprising at least one non-transitory computer-readable medium including program instructions that, when executed by at least one processor, cause the at least one processor to:
receive, from an imaging system including an imager configured to image a patient, for a procedure for the patient, imager protocol data associated with an imager protocol according to which the imaging system is configured to control imaging of the patient with the imager, wherein the imaging system is in communication with an injection system including an injector configured to deliver fluid and/or imaging agent to the patient;
receive, via at least one user interface, for the procedure for the patient, at least one of a selection, a programming, a modification, or any combination thereof of an injector protocol according to which the injection system is configured to control delivery of at least one fluid and/or imaging agent to the patient with the injector; and
associate, in at least one database, the imager protocol with the injector protocol.

22. The computer program product of claim 21, wherein the program instructions, when executed by the at least one processor, further cause the at least one processor to:
determine, from the at least one database, based on the imager protocol data associated with the imager protocol, at least one proposed injector protocol proposed to be used with the imager protocol in the procedure for the patient; and
provide, via the at least one user interface, an option to select, program, or modify the at least one proposed injector protocol proposed for use with the imager protocol in the procedure for the patient.

23. The computer program product of claim 22, wherein the at least one proposed injector protocol is further determined, from the at least one database, based on a number of previous procedures for which the at least one proposed injector protocol was used with the imager protocol.

24. The computer program product of claim 22, wherein the imager protocol data associated with the imager protocol includes at least one procedure specific imager parameter of the following procedure specific imager parameters: a patient name, a patient weight, a tube current level, a tube voltage level, an image acquisition sequence, an identifier of the imager protocol, or any combination thereof.

25. The computer program product of claim 24, wherein the program instructions, when executed by the at least one processor, further cause the at least one processor to:
automatically determine, based on at least one value of the at least one procedure specific imager parameter, at least one value of at least one procedure specific injector parameter associated with the at least one proposed injector protocol.

26. The computer program product of claim 25, wherein the at least one proposed injector protocol includes a smart injector protocol, and wherein automatically determining the at least one value of the at least one procedure specific injector parameter associated with the at least one proposed injector protocol includes calculating, based on the at least one value of at least one procedure specific imager parameter, at least one smart injector protocol parameter of the following smart injector protocol parameters: a flow rate of at least one fluid and/or imaging agent to be delivered to a patient in at least one phase of an injection, a volume of the at least one fluid and/or imaging agent to be delivered to the patient in the at least one phase of the injection, a contrast or imaging agent dose amount, a contrast or imaging agent dose rate, a weight-based parameter, a patient identifier, a patient body surface area, a patient height, a patient body mass index (BMI), a patient sex, a patient cardiac output, a patient anatomy to be imaged, or any combination thereof.

27. The computer program product of claim 25, wherein automatically determining the at least one value of the at least one procedure specific injector parameter associated with the at least one proposed injector protocol includes automatically populating a prompt for the at least one procedure specific injector parameter in the at least one user interface with the determined at least one value of the at least one procedure specific injector parameter.

28. The computer program product of claim 25, wherein the program instructions, when executed by the at least one processor, further cause the at least one processor to:
receive, via the at least one user interface, one or more values of one or more procedure specific injector parameters associated with the at least one proposed injector protocol; and
automatically determine, based on the one or more values of the one or more procedure specific injector parameters, one or more values of one or more procedure specific imager parameters associated with the imager protocol.

29. The computer program product of claim 28, wherein automatically determining the one or more values of the one or more procedure specific imager parameters associated with the imager protocol includes automatically populating a prompt for the one or more procedure specific imager parameters in the at least one user interface with the determined one or more values of the one or more procedure specific imager parameters.

30. The computer program product of claim 25, wherein the at least one user interface provides, in a same display, a plurality of prompts for a plurality of procedure specific imager parameters associated with the imager protocol and a plurality of prompts for a plurality of procedure specific injector parameters associated with the at least one proposed injector protocol.
